(19) United States
(12) Patent Application Publication
(10) Pub. No.: US 2004/0127541 A1
(43) Pub. Date: Jul. 1, 2004

(54) Bicifadine formulation

(76) Inventors: Janet Codd, Athlone (IE); Brian Boland, Maynooth (IE)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/621,435

(22) Filed: Jul. 17, 2003

Related U.S. Application Data
(60) Provisional application No. 60/399,852, filed on Jul. 31, 2002.

(51) Int. Cl. A61K 9/14; A61K 31/403

(52) U.S. Cl. 514/412; 424/488

(57) ABSTRACT

A method and composition form orally administering a unit dosage form composition containing bicifadine and salts thereof, which composition contains controlled release and instant release portions.

### CROSS-REFERENCED TO THE RELATED PROVISIONAL APPLICATION

This application claims the benefit of the U.S. Provisional Application 60/399,852, filed Jul. 31, 2003.

### BACKGROUND OF THE INVENTION

The compounds of the formula

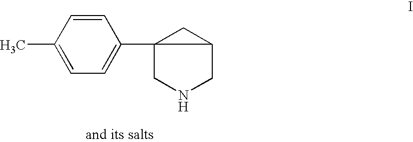

are analgesics that are not narcotics (that is, are not morphine-like in action). See U.S. Pat. No. 4,231,935 and U.S. Pat. No. 4,196,120. The compounds of formula I include bicifadine. In administering the compound of formula I to produce analgesia, it is important that it be administered in such a way that there is a very rapid and strong onset of activity followed by a sustained maintenance of this activity through the presence of this compound in the blood system of the patient. In this manner the patient is kept free from pain. This is especially important with patients suffering from acute pain which results after major surgery and during recovery. It has been desired to develop an analgesic and a method for its delivery that will rapidly relieve moderate and severe pain when administered and will maintain this relief for long periods of time.

### SUMMARY OF THE INVENTION

In accordance with this invention, we have developed a dosage form and a method for delivering the compound of formula I or its salts for relieving pain which quickly relieves pain when administered and maintains this relief for a long period of time. In accordance with the invention it has been found that when the compound of formula I is administered in an effective amount to relieve pain utilizing a dosage regimen of from about 25 mg. to about 600 mg. once or twice daily in an oral unit dosage form containing as its composition this amount of the compound of formula I or its salt, 40% to 60%, by weight of the composition, of a pharmaceutically acceptable carrier and from about 15% to 25% by weight of the composition of a hydroxypropyl methyl cellulose slow release matrix with the carrier and the active ingredient dispersed in the slow release matrix.

### DETAILED DESCRIPTION

This invention is directed to a new unit dosage form and method for administering this dosage form containing the compound of formula I above or its salts to reduce pain in patients suffering such pain. This method produces a strong, rapid onset of relief followed by a sustained maintenance of this relief for a long period of time. The unit oral dosage form of this invention is a sustained release composition containing from about 25 to 600 mg. of the compound of formula I above or its pharmaceutically acceptable salts, a pharmaceutically acceptable carrier in combination with the hydrophilic hydroxypropyl methyl cellulose polymeric matrix. In accordance with this invention it is found that the use of from 15% to 50% by weight, preferably 20% to 25% by weight, based upon the total weight of the composition of hydroxypropyl methyl cellulose polymeric matrix produces a controlled release formulation of the compound of formula I above causing an initial rapid release of this active ingredient in the blood system of the patient to provide an immediate relief of pain and thereafter maintaining a constant slow release of the compound of formula I above for an extended period. In accordance with this invention these beneficial results can be achieved by the administration of oral unit dosage form once or twice daily depending upon the severity of the pain.

The compounds of Formula I include the compound bicifadine and various optical and geometric isomers thereof. The isomers may be in pure form or may be a mixture. The compounds of Formula I include these compounds as well as all forms of these compounds.

In accordance with this invention, the compound of formula I above or its salts are administered in an effective amount to alleviate pain. In general oral dosages of from about 0.5 mg/kg to about 20 mg/kg per day are used. However the amount of the compound of formula I or its salt in the oral unit dose to be administered will depend to a large extent on the amount of pain and the weight of the patient and of course be subject to the physician's judgment. For acute pain which results from invasive surgery, for example, it is best to administer this oral unit dosage form twice a day. On the other hand, for pain resulting from tooth aches, dental or minor surgery once a day administration of this oral dosage form may be sufficient. In accordance with this invention, the oral unit dosage form containing the compound of formula I and/or its salts can be administered at a dosage of from 25 to 600 mg. either once or twice a day. For patients of from about 60 kg. to about 80 kg. unit oral dosage forms containing from about 100 mg. to about 600 mg. can be utilized, with dosages of about 200 to 400 mg. being generally preferred. This oral unit dosage form can be administered once or twice a day. For less pain and for patients whose weight is below 60 kg. an oral unit dosage form containing from about 25 mg to about 200 mg. can be utilized either once or twice a day depending on the patients needs.

In accordance with this invention, it has been found that the beneficial results are achieved through the use of the hydrophilic slow release polymer, hydroxypropyl methyl cellulose. It is this hydrophilic polymer which allows the immediate onset of relief followed by the continued maintenance of the active ingredient in the blood stream of the patient. The hydrophilic slow release hydroxypropyl methyl cellulose polymer that is used in accordance with this invention has a viscosity in the range of about 100 cps to about 100,000 cps. Generally the hydroxypropyl methyl cellulose polymers which are preferred have a viscosity in the range of from about 15,000 cps to about 100,000 cps.

On exposure to aqueous fluids such as in the body of the patient when the oral dosage form such as a tablet is swallowed, the tablet surface becomes wet, and the polymer starts to hydrate to form a gel layer. The soluble nature of the active ingredient causes an initial burst from the external layer of the tablet. Thereafter an expansion of the gel layer occurs when water permeates into the tablet increasing the thickness of the gel layer. The soluble drug diffuses through the gel layer. Concomitantly, the outer layers become fully hydrated and dissolve, a process generally referred to as erosion. Water continues to permeate towards the tablet core until it has dissolved. This initial burst release of the active ingredient should be sufficient to provide a fast onset of action without the need for separate inclusion of an immediate release portion in the dosage form. This polymer provides a release which constitutes an initial burst followed by a continued sustained release of the active ingredient of formula 1 or its salt. In accordance with this invention the composition containing the compound of formula 1 or its salt is released so that not less than 10% of this active ingredient is released within 15 minutes and not less than 50% of this active ingredient is released within 4 hours a and not less than 85% by weight of this active ingredient is released within 12 hours.

The compounds of formula I may be in their acid-addition salt form. The term “pharmaceutically acceptable salts” refers to those acid-addition salts of the parent compound which do not significantly adversely affect the pharmaceutical properties (e.g., toxicity, effectiveness, etc.) of the parent compound such as are conventionally used in the pharmaceutical art. These acid-addition salts are prepared by treatment of the parent compound with the appropriate organic or inorganic acid in a manner well-known to those skilled in the art. The hydrochloride, phosphate, citrate, fumarate, maleate, succinate, pamoate, and sulfate acid-addition salts are preferred. Particularly preferred is the hydrochloride salt. It is to be understood that for the purposes of this invention, the acid-addition salts are equivalent to the parent free base.

In accordance with this invention, the composition in the oral unit dosage form contains a carrier. Suitable carriers include microcrystalline cellulose, lactose, sucrose, fructose, glucose dextrose, or other sugars, di basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol or mixtures thereof. The preferred carrier is di basic calcium phosphate. The diluent or carrier is present in the composition in an amount of about 40% to 60% by weight of the composition

The preferred unit oral dosage form for use in this invention is a tablet. Any conventional method of preparing pharmaceutical oral unit dosage forms can be utilized in preparing the unit dosage forms of this invention. The pharmaceutical oral unit dosage forms, such as the tablets, contain one or more of the conventional additional formulation ingredients. These ingredients are selected from a wide variety of excipients known in the pharmaceutical formulation art. According to the desired properties of the oral dosage form, any number of ingredients may be selected alone or in combination for their known use in preparing such dosage forms as tablets. Such ingredients include, but are not limited to glidants, compression aides, disintegrants, lubricants, binders, flavors, flavor enhancers, sweeteners and preservatives.

Suitable lubricants include stearic acid, magnesium stearate, talc, calcium stearate, hydrogenated vegetable oils, sodium benzoate, sodium chloride, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate.

In accordance with this invention, any conventional means for preparing standard oral unit dosage forms can be utilized. In forming tablets, the blend can be compressed by conventional means to form the tablets of this invention. The term “tablet” as used herein is intended to encompass compressed pharmaceutical dosages formulations of all sizes and shapes whether coated or uncoated. Substances which may be used for coating include hydroxypropyl cellulose, titanium oxide, talc, sweeteners and colorants.

The invention is further illustrated by the following examples.

### IN THE EXAMPLES

Bicifadine HCI is the hydrochloric acid salt of the compound of formula I.

Emcompress is the carrier dibasic calcium phosphate.

Methocel K100M is the hydrophilic polymeric hydroxypropyl methyl cellulose having a viscosity of 100,000 cps for a 2% solution in water [HPMC].

Methocel K100LV is the hydrophilic polymeric hydroxypropyl methyl cellulose having a viscosity of 100 cps for a 2% solution in water [HPMC].

Carbopol 971P is a polyacrylic acid polymer having a viscosity of 4,000 to 12,000 cps for a 0.5% solution at pH 7.5[PAA].

Aerosil 200 is colloidal silicon dioxide.

Avicel PH101 is microcrystalline cellulose.

The content of the active ingredient of formula I in the sample as reported in the dissolution tables was determined by HPLC.

### Example 1

### Preparation of 200 mg. Bicifadine HCI Tablet

Bicifadine HCI 200 mg.-slow release tablet were prepared using the following ingredients. In the table below the “% composition” is the % by weight of the ingredient based upon the total weight of the composition.

(i) Bicifadine HCI 200 mg SR Tablets

Batch Size: 5.2 kg

| Material | % Composition | Mg/tablet |
|---|---|---|
| Bicifadine HCI | 31.25 | 200.0 |
| Methocel K100M | 20.00 | 128.0 |
| Emcompress | 47.75 | 305.6 |
| Magnesium Stearate | 0.50 | 3.2 |
| Aerosil 200 | 0.50 | 3.2 |

The tablets are prepared from the above ingredients set forth below.

(1) Sieve the Bicifadine HCI through a 1 mm screen, and collect in a polyethylene lined container. Weigh the exact quantity required.

(2) Add the Aerosil 200 to a portion of the Emcompress. Bag blend for 2 minutes and pass through a 600 micron screen.

(3) Add the Magnesium Stearate to a portion of the Emcompress. Bag blend for 2 minutes and pass through a 600 micron screen.

(4) Transfer the components to a V cone blender (Pharmatech Mobile Multi-Blend Blender, equipped with 25 L V cone), and blend for 20 minutes at 18 rpm.

Order of Addition:

Half of Emcompress

Sieved Emcompress/Aerosil mix

Sieved Bicifadine HCI

Methocel K100M

Remaining Emcompress

(5) Add the Sieved Emcompress/Magnesium Stearate mix, and blend for a further 3 minutes at 18 rpm.

(6) Tablet the blend using a rotary tablet press (Piccola Tablet Press)

Tabletting Parameters

| | |
|---|---|
| Press Speed Setting: | 6 |
| Punch Description: | 18 × 8 mm oval normal concave |
| No of punches: | 5 |
| Main Compression Force Setting: | 2.5 |
| Filomatic Speed Setting | 4 |
| Target Tablet Weight: | 0.640 g (Range: 0.595-0.685 g) |
| Target Tablet Hardness | 150 N (Range: 105-195 N) |

### Example 2

Preparation of 200 mg. Bicifadine HCI Tablet

Bicifadine HCI 200 mg slow release tablets were prepared using the following ingredients. In the table below the “% composition” is the % by weight of the ingredient based upon the total weight of the composition.

Bicifadine HCI 200 mg SR Tablets

| Material | % Composition | Mg/tablet |
|---|---|---|
| Bicifadine HCI | 31.25 | 200.0 |
| Methocel K100M | 40.00 | 256.0 |
| Emcompress | 27.25 | 174.4 |
| Magnesium Stearate | 01.00 | 006.4 |
| Aerosil 200 | 00.50 | 003.2 |

The Bicifadine HCl sustained release tablets were manufactured similarly to Example 1.

### Example 3

Preparation of 200 mg. Bicifadine HCI Tablet

Bicifadine HCI 200 mg slow release tablets were prepared using the following ingredients. In the table below the “% composition” is the % by weight of the ingredient based upon the total weight of the composition.

Bicifadine HCI 200 mg SR Tablets

| Material | % Composition | Mg/tablet |
|---|---|---|
| Bicifadine HCl | 31.25 | 200.0 |
| Methocel K100M | 60.00 | 384.0 |
| Emcompress | 07.25 | 046.4 |
| Aerosil 200 | 00.50 | 003.2 |
| Magnesium Stearate | 01.00 | 006.4 |

The Bicifadine HCl sustained release tablets were manufactured similarly to Example 1.

### Example 4

### Dissolution of 200 mg. Bicifadine HCI Tablet

Dissolution Testing of Examples 1, 2 and 3 was performed using USP 1 Apparatus, 20 mesh baskets, 75 rpm, 900 ml phosphate buffer pH 6.8±0.05, 37° C.±0.5° C.

| | Example 1 | Example 2 | Example 3 |
|---|---|---|---|
| Time (Hrs) | Mean % Released | | |
| 0.25 | 14.6 | 11.2 | 9.2 |
| 0.5 | 22.9 | 16.8 | 13.1 |
| 1 | 33.5 | 24.0 | 21.1 |
| 2 | 48.4 | 37.3 | 33.2 |
| 4 | 69.1 | 54.4 | 48.4 |
| 8 | 89.7 | 76.8 | 69.7 |
| 12 | 99.9 | 88.4 | 82.7 |
| 22 | — | 100.6 | 95.5 |

For these tablets, a substantial amount of the active ingredient is released at the early timepoints. For Example 1 in particular, a significant portion of the total amount of active ingredient (˜15%) is released within the first 15 minutes, with the remainder released in a slow and continuous manner over the remaining 12 hrs.

### Example 5

### Preparation of 200 mg. Bicifadine HCI Tablet

Bicifadine HCI 200 mg slow release tablets were prepared using the following ingredients. In the table below the “% composition” is the % by weight of the ingredient based upon the total weight of the composition.

| Material | % Composition | Mg/tablet |
|---|---|---|
| Bicifadine HCl | 31.25 | 200.00 |
| Methocel K100M | 30.00 | 192.00 |
| Emcompress | 37.75 | 241.60 |
| Aerosil 200 | 00.50 | 003.20 |
| Magnesium Stearate | 00.50 | 003.20 |

The Bicifadine HCl sustained release tablets were manufactured similarly to Example 1.

### Example 6

### Preparation of 200 mg. Bicifadine HCI Tablet

Bicifadine HCI 200 mg slow release tablets were prepared using the following ingredients. In the table below the “% composition” is the % by weight of the ingredient based upon the total weight of the composition.

| Material | % Composition | Mg/tablet |
|---|---|---|
| Bicifadine HCl | 31.25 | 200.00 |
| Methocel K100M | 13.60 | 087.04 |
| Methocel K100LV | 26.40 | 168.96 |
| Emcompress | 27.75 | 177.60 |
| Aerosil 200 | 00.50 | 003.20 |
| Magnesium Stearate | 00.50 | 003.20 |

The Bicifadine HCl sustained release tablets were manufactured similarly to Example 1.

### Example 7

### Dissolution of 200 mg. Bicifadine HCI Tablet

Dissolution Testing of Examples 5 and 6 was performed using USP 1 Apparatus, 20 mesh baskets, 75 rpm, 900 ml phosphate buffer pH 6.8±0.05, 37° C.±0.5° C.

| | |
|---|---|
| Example 5 | Example 6 |

| | |
|---|---|
| Time (Hrs) | Mean % Released |

| | | |
|---|---|---|
| 0.25 | 13.9 | 13.3 |
| 0.5 | 21.6 | 19.2 |
| 1 | 28.3 | 27.7 |
| 2 | 41.8 | 41.4 |
| 4 | 60.7 | 60.4 |
| 8 | 85.3 | 85.5 |
| 12 | 96.1 | 97.4 |
| 22 | 104.1 | 101.0 |

### Example 8

### Preparation of 180 mg. Bicifadine HCI Tablet

Bicifadine HCI 180 mg slow release tablets were prepared using the following ingredients. In the table below the “% composition” is the % by weight of the ingredient based upon the total weight of the composition.

| | A | B | C | D | E | F |
|---|---|---|---|---|---|---|
| Material | % Composition | | | | | |
| Bicifadine HCI | 30.0 | 30.0 | 30.0 | 30.0 | 40.0 | 40.0 |
| Methocel | 30.0 | — | 30.0 | — | 30.0 | 40.0 |
| K100M | | | | | | |
| Methocel | — | 30.0 | — | 30.0 | — | — |
| K15M | | | | | | |
| Emcompress | — | — | 38.5 | 38.5 | — | — |
| Pharmatose | 38.5 | 38.5 | — | — | — | — |
| DCL 11 | | | | | | |
| Mannitol | — | | — | — | 23.5 | 18.5 |
| Aerosil 200 | 00.5 | 00.5 | 00.5 | 00.5 | 00.5 | 00.5 |
| Magnesium | 01.0 | 01.0 | 01.0 | 01.0 | 01.0 | 01.0 |
| Stearate | | | | | | |
| Tablet Weight | 600 mg | 600 mg | 600 mg | 600 mg | 450 mg | 450 mg |

The blend was manufactured using manual blending. Tablets were compressed manually using 300 bar pressure and an Enerpac single station tablet press using 13 mm normal concave tooling.

### Example 9

### Dissolution of 180 mg. Bicifadine HCI Tablet

Dissolution Testing of Example 8 was performed using USP 2 Apparatus, 50 rpm, 900 ml phosphate buffer pH 6.8±0.05, 37° C.±0.5° C.

| | A | B | C | D | E | F |
|---|---|---|---|---|---|---|
| Time (Hrs) | Mean % Released | | | | | |
| 0.25 | 17.7 | 17.2 | 16.8 | 21.0 | 18.9 | 17.8 |
| 1 | 25.5 | 24.9 | 24.4 | 30.7 | 27.1 | 22.7 |
| 4 | 52.3 | 51.0 | 48.7 | 57.4 | 54.1 | 54.3 |
| 8 | 74.3 | 70.3 | 66.0 | 73.2 | 74.3 | 75.0 |
| 12 | 88.6 | 84.4 | 77.2 | 84.1 | 87.8 | 89.0 |
| 22 | 101.4 | 99.3 | 91.1 | 96.5 | 99.8 | 100.8 |

### Example 10

### Preparation of 200 mg Bicifadine HCL Tablet

This Example is directed to the preparation of 200 mg Bicifadine HCL tablets which contain another slow release polymer such as polyacrylic acid polymer alone (Example 10A) or combined with hydroxypropylmethyl cellulose (Example 10B).

Bicifadine HCl 200 mg slow release tablets were prepared using the following ingredients. In the table below, the “% composition” is the % by weight of the ingredient based upon the total weight of the composition.

| | A | | B | |
|---|---|---|---|---|
| | % | Amount | % | Amount |
| Material | Composition | mg/tab | Composition | mg/tab |
| Bicifadine HCl | 31.25 | 200 | 31.25 | 200 |
| Carbopol 971P | 15.0 | 96 | 10.0 | 64 |
| Methocel K100M | — | — | 40.0 | 256 |
| Emcompress | 52.25 | 334.4 | 17.25 | 110.4 |
| Aerosil | 0.5 | 3.2 | 0.5 | 3.2 |
| Magnesium Stearate | 1.0 | 6.4 | 1.0 | 6.4 |

The Bicifadine HCl tablets were manufactured similarly to Example 1, with Carbopol 971P substituting Methocel K100M as required. The target tablet hardness was 200N (Range: 140-260N).

### Example 11

### Dissolution of 200 mg Bicifadine HCL Tablets of Examples 10A and 10B

Dissolution Testing of Example 10(A) and 10(B) was performed using USP 1 Apparatus, 20 mesh baskets, 75 rpm. The dissolution medium used was 900 ml 0.01N HCl for the first two hours, followed by 900 ml phosphate buffer pH 6.8±0.05, 37° C.±0.5° C. for the remaining time.

| | |
|---|---|
| Example 10(A) | Example 10(B) |

| | |
|---|---|
| Time (hrs) | Mean % Released |

| | | |
|---|---|---|
| 0.25 | 17.6 | 12.0 |
| 0.5 | 23.6 | 16.7 |
| 1 | 31.2 | 22.9 |
| 2 | 42.9 | 32.8 |
| 4 | 49.9 | 42.9 |
| 8 | 59.7 | 58.1 |
| 12 | 65.7 | 67.4 |
| 22 | 74.2 | 81.2 |

### Example 12

### Preparation of 100 mg Bicifadine HCL Tablet

This example is directed to the preparation of Bicifadine HCl 100 mg rapid release tablets which do not contain any hydrophilic slow release polymer matrix much less a hydroxypropylmethyl cellulose. These tablets were prepared for use as a control. Bicifadine HCl 100 mg rapid release tablets were prepared using the following ingredients. In the table below, the “% composition is the % by weight of the ingredient based upon the total weight of the composition.

| Material | % Composition | Mg/tab |
|---|---|---|
| Bicifadine | 15.625 | 100 |
| Avicel PH101 | 72.875 | 466.4 |
| Polyplasdone | 10.0 | 64 |
| Aerosil | 0.5 | 6.4 |
| Magnesium Stearate | 1.0 | 3.2 |

The tablets are prepared from the above ingredients as set forth below.

(1) Blend Avicel PH101 with Aerosil 200 in a ratio of ca. 1:40 for two minutes, then pass through a screen of aperture 600Tm.

(2) Blend Avicel PH101 with Magnesium Stearate in a ratio of ca. 1:20 for two minutes, then pass through a screen of aperture 600Tm.

(3) Pass Bicifadine raw material through a 1 mm screen. Weigh the exact amount required.

(4) Transfer the components to a V cone blender (Pharmatech Mobil Multi-Blend Blender),

Order of addition equipped with 25 L cone, and blend for ten minutes at 18 rpm

Approximately half of the remaining Avicel PH101

Polyplasdone

Screened Avicel/Aerosil blend to blender.

Remaining Avicel to the blender.

(5) Add the screened Avicel/Magnesium Stearate to the blender and blend for three minutes at 18 rpm.

Tablet the blend using a rotary tablet press (Piccola Tablet Press), using 18×8 mm oval normal concave tooling to a target Tablet Weight of 0.640 g (Range: 0.595-0.685 g).

### Example 13

### Dissolution of 100 mg Bicifadine HCL Tablets of Example 12

Dissolution Testing of Example 12 was performed using USP 1 Apparatus, 20 mesh baskets, 75 rpm. The dissolution medium used was 900 ml 0.01N HCl, 37° C.±0.5° C.

| | Example 12 |
|---|---|
| Time (Hrs) | % Released |
| 0.083 | 95.6 |
| 0.5 | 101.1 |

### Example 14

### In Vivo Pharmacokinetic Study

This example is to demonstrate that the use of Bicifadine, oral dosage forms having from about 20-50% by weight of hydroxypropylmethyl cellulose hydrophilic slow release polymer matrix produces a sustained maintenance of Bicifadine in the blood for longer periods of time than utilizing comparable matrix systems which contain greater than 50% hydroxypropylmethyl cellulose as well as systems which contain other sustained release polymer matrixes.

In this study the following treatments were evaluated: 1) Treatment A=Tablets of Example 12, no slow release; 2) Treatment B=Tablets of Example 2 (40% HPMC); 3) Treatment C=Tablets of Example 3 (60% HPMC); 4) Treatment D=Tablets of Example 10B (40% HPMC and 10% PAA); and 5) Treatment E=Tablets of Example 10A (PAA).

A five treatment, randomized balanced crossover study in 15 healthy volunteers examining the absorption of Bicifadine HCl sustained release tablets relative to an rapid release tablet was performed as follows:

Study:

A five treatment, randomized balanced crossover study in healthy volunteers examining the absorption of various (Bicifadine) sustained release tablets relative to rapid release tablet and evaluating the safety and tolerability of the test compounds administered orally.

Objective:

To evaluate the effect of different types/levels of matrix-forming polymers within sustained release tablets

To evaluate the release of bicifadine from rapid release tablets

To evaluate the safety and tolerability of the test compounds administered orally

Methodology:

Five-Treatment, 5-period, fasted, balanced crossover study with a three to four day washout between each dose.

Number of Subjects:

Fifteen (15) healthy volunteers.

Diagnosis and Main Criteria for Inclusion:

Healthy male volunteers, aged greater than 18 and less than 40 years, and within ±10% of ideal body weight.

Duration of Treatment:

The test treatment was administered as a single oral dose. In each treatment period the duration of stay in the clinic was approximately 12 hours prior to dosing and 24 hours after dosing. There was 5 treatment periods. There was a 3-4 day washout period between each dose administration (for example, a Monday/Thursday or equivalent dosing schedule).

The total duration of the study was approximately 28 days. Total confinement during the study was 10 days and 10 nights.

During each day of the 28 day period the blood of each of the patients was extracted and the concentration of Bicifadine in the blood was evaluated and analyzed and reported in ng/ml. The concentration of ng/ml of drug in the plasma was plotted against time and various features of the resulting curve were measured and reported in the table as follows.

Abbreviations:

Area under the drug plasma concentration versus time curve=AUCO-t.

Area under the drug plasma concentration versus time curve extrapolated to infinity=AUCO-int.

The maximum measured concentration of the drug in the plasma=Cmax.

The time at which the Cmax was measured=tmax.

Terminal elimination rate=Lamda_z.

Apparent half life=t½.

| | | | | TrtD - | |
|---|---|---|---|---|---|
| | | TrtB - | TrtC - | 200 mg | |
| | TrtA - | 200 mg | 200 mg | Bicifadine SR | Trt E |
| | 100 mg | Bicifadine SR | Bicifadine SR | (40% Methocel | 200 mg |
| | Bicifadine IR | (40% Methocel | (60% Methocel | K100M and 10% | Bicifadine SR |
| PK | control tablet | K100M) | K100M) | Carbopol) | (15% Carbopol) |
| Parameters | n15 | n15 | n15 | n15 | n15 |
| AUCinf | 2621.81 ± 838.33 | 4837.19 ± 1801.19† | 3506.81 ± 1819.09* | 3764.95 ± 1538.40† | 3160.12 ± 2071.62‡ |
| (ng/mL · h) | | | | | |
| CV % | 32.0 | 37.2 | 51.9 | 40.9 | 65.6 |
| AUClast | 2578.75 ± 805.08 | 4460.36 ± 1390.56 | 3293.94 ± 1372.03 | 3273.54 ± 995.39 | 3308.39 ± 1573.14 |
| (ng/mL · h) | | | | | |
| CV % | 31.2 | 31.2 | 41.7 | 30.4 | 47.6 |
| Cmax | 1485.93 ± 495.32 | 546.36 ± 103.69 | 440.35 ± 81.74 | 545.58 ± 165.75 | 398.82 ± 125.89 |
| (ng/mL) | | | | | |
| CV % | 33.3 | 19.0 | 18.6 | 30.4 | 31.6 |
| Tmax | 0.53 ± 0.26 | 1.47 ± 0.90 | 1.50 ± 0.93 | 0.80 ± 0.44 | 1.52 ± 0.91 |
| (h) | | | | | |
| CV % | 47.9 | 61.1 | 61.7 | 55.4 | 59.8 |
| Lambda_z | 0.41 ± 0.13 | 0.16 ± 0.09† | 0.22 ± 0.13* | 0.11 ± 0.08† | 0.20 ± 0.10‡ |
| (h⁻¹) | | | | | |
| CV % | 31.1 | 54.0 | 61.8 | 73.1 | 51.3 |
| t1/2 | 1.84 ± 0.56 | 5.55 ± 2.49† | 4.74 ± 3.38* | 9.36 ± 4.63† | 4.96 ± 3.36‡ |
| (h) | | | | | |
| CV % | 30.8 | 44.8 | 71.2 | 49.5 | 67.8 |

*n = 14
† n = 12
‡ n = 9

From the plotted plasma profiles for each of the treatments, and the pharmacokinetic parameters reported in the table, the tablets which contained 40% by weight hydroxymethyl cellulose had a higher concentration of drug in the blood stream for longer periods of time than those produced from tablets containing 60% hydroxypropyl methylcellulose slow release polymer matrix. This was clearly observed by comparing Treatment B with Treatment C. In addition, Treatment B which contained 40% by weight of hydroxypropylmethyl cellulose hydrophilic slow release polymer matrix produced far superior results with regard to the maintenance of Bicifadine in the blood stream for longer periods of time than that produced Treatment E by the tablets containing either polyacrylic acid alone as the slow release polymer matrix or in a mixture with hydroxypropyl cellulose (Treatment D).

What is claimed is:

1. A method for reducing pain in a patient in need of said treatment comprising orally administering to said patient in a unit oral dosage form a composition containing from about 25 to 600 mg. of an active ingredient selected from the group consisting of a compound of the formula 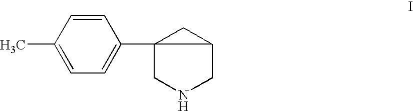
or a pharmaceutically acceptable salt,
a pharmaceutically acceptable carrier in an amount of from about 40% to 60% of weight of said composition, and from about 15% to 50% by weight, of said composition of, a hydroxypropyl methyl cellulose hydrophilic slow release polymer matrix, said unit dosage being orally administered to said patient from once to twice a day.

2. The method of claim 1 wherein said dosage form is a tablet.

3. The method of claim 2, wherein the polymer matrix hydroxypropyl methyl 2 cellulose is present in an amount of from about 20% to 40% by weight of the composition.

4. The composition of claim 3 wherein said polymer matrix has a viscosity of from 2 about 100 to about 100,000 cps.

5. The method of claim 2 wherein the carrier is dibasic calcium phosphate.

6. The method of claim 5 wherein the active ingredient is present in the unit dosage form in an amount of about 150-400 mg.

7. The method of claim 1 wherein the patient is suffering from acute pain and the unit dosage form is administered once or twice a day.

8. The method of claim 7 where the patient is suffering from minor pain and the unit dosage form is administered once a day.

9. A unit oral dosage form comprising a composition containing from about 25 to 600 mg. of an active ingredient selected from the group consisting of a compound of the formula 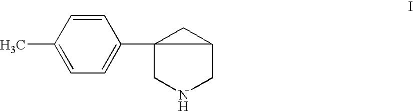
or a pharmaceutically acceptable salt,
from about 40% to 60% of weight of said composition of pharmaceutically acceptable carrier and from about 15% to about 50% of weight of said composition of a hydroxypropyl methyl cellulose hydrophilic slow release polymer matrix.

10. The unit oral dosage form of claim 9 wherein said composition is in the form of a tablet.

11. The unit dosage form of claim 9 wherein the hydroxypropyl methyl cellulose polymer matrix is present in an amount of from about 20% to 40% by weight of this composition.

12. The unit dosage form of claim 9 wherein said polymer matrix has a viscosity of from about 100 to about 100,000 cps.

13. The unit dosage form of claim 10 wherein said active ingredient is present in an amount of 200 mg.
